(19) United States
(12) Patent Application Publication
(10) Pub. No.: US 2024/0216472 A1
(43) Pub. Date: Jul. 4, 2024

(54) ENHANCED EFFICACY OF TOLEROGENIC VACCINATION

(71) Applicant: Therapeutic Solutions International, Inc., Oceanside, CA (US)

(72) Inventors: Thomas E. ICHIM, Oceanside, CA (US); Kalina O'CONNOR, Oceanside, CA (US); James VELTMEYER, Oceanside, CA (US); Timothy G. DIXON, Oceanside, CA (US)

(73) Assignee: Therapeutic Solutions International, Inc., Oceanside, CA (US)

(21) Appl. No.: 18/404,724

(22) Filed: Jan. 4, 2024

Related U.S. Application Data
(60) Provisional application No. 63/437,096, filed on Jan. 4, 2023.

(51) Int. Cl. A61K 38/20; A61K 9/00; A61K 38/17; A61K 45/06; A61P 37/06

(52) U.S. Cl. CPC A61K 38/2066; A61K 9/0019; A61K 38/1709; A61K 45/06; A61P 37/06

(57) ABSTRACT

Disclosed are means, methods, and compositions of matter useful for induction of antigen specific suppression of immunity and/or tolerogenesis through administration of tolerogenic agents together with antigens and/or modified antigens delivered via multiple intradermal injections. In one embodiment the invention teaches the use of a tattoo gun or a similar device to administer over an extended area of skin a compound which induces a tolerogenic microenvironment and subsequently administration of said antigen in the artificially created microenvironment. The essence of the disclosed invention is the superior tolerogenic effects observed when tolerogenic stimuli and antigen are administered over an extended area of skin through the use of a tattoo gun or similar device

### CROSS-REFERENCE TO RELATED APPLICATIONS

This application claims the benefit of priority to U.S. Provisional Application Ser. No. 63/437,096, filed on Jan. 4, 2023, the entire contents of which are incorporated herein by reference.

### BACKGROUND OF THE INVENTION

Immunological tolerance is a cardinal feature of the immune system, allowing for recognition and elimination of pathological threats, while selectively ignoring antigens that belong to the body. Understanding mechanisms of immunological tolerance, and having the ability to induce this process would make a major impact in autoimmune conditions, which affect approximately 8% of the US population.sup.1. Major autoimmune diseases include rheumatoid arthritis, multiple sclerosis, type 1 diabetes, systemic lupus erythromatosis, and inflammatory bowel disease. Traditionally, autoimmune conditions are treated with non-specific inhibitors of inflammation such as steroids, as well as immune suppressive agents such as cyclosporine, 5-azathrioprine, and methotrexate. These approaches globally suppress immune functions and have numerous undesirable side effects. Unfortunately, given the substantial decrease in quality of life observed in patients with autoimmunity, the potential of alleviation of autoimmune symptoms outweighs the side effects such as opportunistic infections and increased predisposition to neoplasia. The introduction of “biological therapies” such as anti-TNF-alpha antibodies has led to some improvements in prognosis, although side effects are still present due to the non-specific nature of the intervention. Regardless, sales of TNF-alpha inhibitors have been quite successful: Humira ($9.2B; 2012), Enbrel ($7.8B; 2011), Remicade ($6.7B; 2011).sup.2. These approaches do not “cure” autoimmunity, but merely alleviate symptomology.

To “cure” autoimmunity, it is essential to delete/inactivate the T cell clone that is recognizing the autoantigen in a selective manner. This would be akin to recapitulating the natural process of tolerance induction. While thymic deletion was the original process identified as being responsible for selectively deleting autoreactive T cells, it became clear that numerous redundant mechanisms exist that are not limited to the neonatal period. Specifically, a “mirror image” immune system was demonstrated to co-exist with the conventional immune system. Conventional T cells are activated by self-antigens to die in the thymus and conventional T cells that are not activated receive a survival signal [1]; the “mirror image”, T regulatory (Treg) cells are actually selected to live by encounter with self-antigens, and Treg cells that do not bind self antigens are deleted [2, 3]. Thus the self-nonself discrimination by the immune system occurs in part based on self antigens depleting autoreactive T cells, while promoting the generation of Treg cells. An important point for development of an antigen-specific tolerogenic vaccine is that in adult life, and in the periphery, autoreactive T cells are “anergized” by presentation of self-antigens in absence of danger signals, and autoreactive Treg are generated in response to self antigens. Although the process of T cell deletion in the thymus is different than induction of T cell anergy, and Treg generation in the thymus, results in a different type of Treg as compared to peripheral induced Treg, in many aspects, the end result of adult tolerogenesis is similar to that which occurs in the neonatal period.

Unfortunately, to date, although some tolerogenic strategies have shown some limited signals of efficacy, there are no effective means of inducing antigen specific tolerance. The current invention seeks to overcome this through creating a localized tolerogenic environment using specific anatomical delivery of tolerogenic agents and antigens.

### BRIEF DESCRIPTION OF THE DRAWINGS

FIG. 1 is a bar graph showing the effects of myelin basic protein and IL-10 on encephalopathy modeled mice.

### DESCRIPTION OF THE INVENTION

As used herein the term “tattoo” does not require coloring of the skin, but is a process similar to the decorative tattooing process wherein color or design is imparted to the skin. Similar devices may be used for decorative tattooing as are used in the present disclosure as will be described herein below. Tattoo as used herein is the process of delivery of one or more bioactive agents to a desired layer of the skin, optionally the dermis, where the bioactive agent may be administered alone, as part of a solution or suspension, and/or with a polymeric moiety or in other form (e.g. salt) so as to increase the residence time of the bioactive agent in the skin.

Administration by tattoo differs from epidermal delivery as it is previously known. For example, epidermal delivery or other prior delivery mechanisms involve making punctures over the surface of the skin and then spreading or rubbing a topical formulation on the perforated skin. The punctures will have no negative pressure. Quite the contrary, body fluids expelled by the punctures exhibit a pressure higher than atmospheric pressure. As such, by these prior processes only topical medications could permeate the holes. Tattooing as used herein is a form of direct dermal delivery (in some aspects). A solid (e.g. sterile metal needle) perforates through the epidermis to the dermis, and is removed at high speed. This removal creates a negative pressure formed in the space created by the needle. This pressure sucks down any fluid located over the hole just formed. This means that if a liquid layer (e.g. droplet, present on or within a needle, etc.) is present above the micro puncture, the liquid and any material carried in the liquid (e.g. bioactive agent/polymeric moiety) will be sucked into the hole and delivered efficiently to the desired skin layer, illustratively the dermis. This mechanism also avoids inadvertent intravascular injections because the pressure is stabilized once the fluid reaches the dermis.

The invention provides the previously undisclosed finding that tattoo gun based immunization is capable of inducing enhanced tolerogenic responses as compared to other types of immunization. In one embodiment tattoo based immunization is utilized together with one or more inhibitors of dendritic cell maturation in order to induce antigen specific suppression of an immune response. To accomplish such a suppression said antigen may be administered prior to, concurrently with, or subsequently to administration of tolerogenic substances. Said tolerogenic substances may directly or indirectly suppress dendritic cell activation. In one embodiment said antigen whose suppression is desired is one or more autoantigens. Said autoantigens are known in the art and can be administered in native or altered form. Suppressors of dendritic cell maturation include interleukin-10, interleukin 1 receptor antagonist, HGF-1 and GM-CSF. Other suppressors are inhibitors of NF-kappa B.

In one embodiment of the invention, immature dendritic cells are utilized to allow for augmentation of endothelial anti-thrombotic functions after a patient receives paclitaxel. In one specific embodiment paclitaxel is given to an ovarian failure patient and immature dendritic cells are administered to reduce potential thrombosis. In another embodiment, immature dendritic cells for patients with ovarian failure. Studies have shown that tissue factor pathway inhibitor expression was reduced by prolonged treatment with either paclitaxel or TNF-alpha [43]. In one embodiment of the invention, immature dendritic cells are administered to increase expression of tissue factor pathway inhibitor expression.

In one aspect of the invention, immature dendritic cells are utilized as biological regulator of inflammation. Under normal conditions, inflammation is a protective response by an organism to fend off an invading agent. Inflammation is a cascading event that involves many cellular and humoral mediators. On one hand, suppression of inflammatory responses can leave a host immunocompromised; however, if left unchecked, inflammation can lead to serious complications including chronic inflammatory diseases (e.g. asthma, psoriasis, arthritis, rheumatoid arthritis, multiple sclerosis, inflammatory bowel disease and the like), septic shock and multiple organ failure. Importantly, these diverse disease states share common inflammatory mediators, such as cytokines, chemokines, inflammatory cells and other mediators secreted by these cells. In the context of the current invention immature dendritic cells are utilized to inhibit pathological inflammation while allow various aspects of the immune response to remain intact.

In one embodiment of the invention immature dendritic cells are utilized to treat ovarian failure.

In some embodiments of the invention immature dendritic cells are administered into the ovary together with mesenchymal stem cells. “Mesenchymal stem cell” or “MSC” in some embodiments refers to cells that are (1) adherent to plastic, (2) express CD73, CD90, and CD105 antigens, while being CD14, CD34, CD45, and HLA-DR negative, are of autologous and/or allogencic origin, and (3) possess ability to differentiate to osteogenic, chondrogenic and adipogenic lineage. Other cells possessing mesenchymal-like properties are included within the definition of “mesenchymal stem cell”, with the condition that said cells possess at least one of the following: a) regenerative activity; b) production of growth factors; c) ability to induce a healing response, either directly, or through elicitation of endogenous host repair mechanisms. As used herein, “mesenchymal stromal cell” or ore mesenchymal stem cell can be used interchangeably. Said MSCcan be derived from any tissue including, but not limited to, bone marrow, adipose tissue, amniotic fluid, endometrium, trophoblast-derived tissues, cord blood, Wharton jelly, placenta, amniotic tissue, derived from pluripotent stem cells, and tooth. In some definitions of “MSC”, said cells include cells that are CD34 positive upon initial isolation from tissue but are similar to cells described about phenotypically and functionally. As used herein, “MSC” may includes cells that are isolated from tissues using cell surface markers selected from the list comprised of NGF-R, PDGF-R, EGF-R, IGF-R, CD29, CD49a, CD56, CD63, CD73, CD105, CD106, CD140b, CD146, CD271, MSCA-1, SSEA4, STRO-1 and STRO-3 or any combination thereof, and satisfy the ISCT criteria either before or after expansion. Furthermore, as used herein, in some contexts, “MSC” includes cells described in the literature as bone marrow stromal stem cells (BMSSC), marrow-isolated adult multipotent inducible cells (MIAMI) cells, multipotent adult progenitor cells (MAPC), mesenchymal adult stem cells (MASCS), MultiStem®, Prochymal®, remestemcel-L, Mesenchymal Precursor Cells (MPCs), Dental Pulp Stem Cells (DPSCs), PLX cells, PLX-PAD, AlloStem®, Astrostem®, Ixmyelocel-T, MSC-NTF, NurOwn™, Stemedyne™-MSC, Stempeucel®, StempeucelCLI, StempeucelOA, HiQCell, Hearticellgram-AMI, Revascor®, Cardiorel®, Cartistem®, Pneumostem®, Promostem®, Homeo-GH, AC607, PDA001, SB623, CX601, AC607, Endometrial Regenerative Cells (ERC), adipose-derived stem and regenerative cells (ADRCs).

Preferred unit dosage formulations are those containing a daily dose or unit, daily sub-dose or an appropriate fraction thereof, of an active ingredient. It is preferred that doses for topical administration of the antagonists of the invention may be of the order of fractions of or multiple mg/kg body weight of the patient. For example, the dose may be between 0.01 to 500 mg/kg body weight; 1 to 400 mg/kg body weight; 2 to 200 mg/kg body weight; 3 to 100 mg/kg body weight or 4 to 50 mg/kg (or any combination of these upper and lower limits, as would be appreciated by the skilled person). The dose used may in practice be limited by the solubility of the compound. Examples of possible doses are 0.01, 0.05, 0.075, 0.1, 0.2, 0.5, 0.7, 1, 2, 5, 10, 12, 15, 20, 25, 30, 35, 40, 45, 50 or 100 mg per kg body weight up to, for example 500 mg/kg body weight, or any value in between. It is envisaged that preferred doses of antagonist would be adjusted according to relative potency. The physician or veterinary practitioner will be able to determine the required dose in a given situation based on the teaching and Examples provided herein.

### Example 1

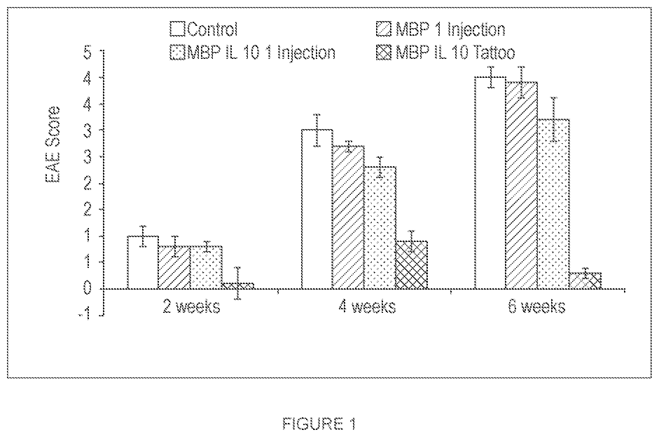

BALB/c mice were immunized with bovine myelin basic protein and complete freunds adjuvant to induce experimental autoimmune encephalopathy on day zero. Control mice receive 0.1 ml saline intradermally, MPB 1 received 10 ug of myelin basic protein intradermally at day 5, MBP IL 1-received 10 ug MBP and 100 ng IL-10 in one intradermal injection, MBP IL 10 tattoo received MPB and IL 10 via tattoo gun over a surface area of 0.5 cm squared. Pathology was quantified on classical EAE score by blinded observer. Results are shown in FIG. 1.

1. A method of modulating an ongoing immune response in an antigen-specific manner through the steps of: a) obtaining one or more tolerogenic agents; b) administering said tolerogenic agents intradermally through multiple injections; c) obtaining one or more chemical compositions representing the antigen to which modulation of immune response is desired; and d)) administering said tolerogenic agents intradermally through multiple injections.

2. The method of claim 1, wherein said tolerogenic agent is selected from a group comprising: a) a small molecule; b) a cytokine; c) a cell; and d) a nucleic acid.

3. The method of claim 2, wherein said small molecule is selected from a group comprising of; a) PGE2; b) n-acetylcysteine; c) resveratrol; d) vitamin D3; and e) kynurenine.

4. The method of claim 2, wherein said cytokine is selected from a group comprising of: a) IL-4; b) IL-10; c) IL-13; d) IL-20; e) IL-35; f) GM-CSF; g) VEGF; h) TGF-beta and i) HGF-1.

5. The method of claim 2, wherein said cell is selected from a group comprising of; a) a M2 monocyte; b) an immature dendritic cell; c) a mesenchymal stem cell; d) a Treg cell; e) a type 2 neutrophil; f) a Th2 cell; g) a Th3 cell; h) a type 2 NKT cell; i) a gamma delta T cell; and j) a B regulatory cell.

6. The method of claim 2, wherein said nucleic acid is either mRNA or DNA encoding a molecule selected from a group comprising of: a) IL-4; b) IL-10; c) IL-13; d) IL-20; e) IL-35; f) GM-CSF; g) VEGF; h) TGF-beta and i) HGF-1.

7. The method of claim 2, wherein said nucleic acid is either mRNA or DNA encoding a molecule selected from a group comprising of: a) PD=L1; b) HLA-G; c) indolamine 2,3 dioxygenase; and e) arginase.

8. The method of claim 1, wherein said antigen is selected from a group comprising of; a) GAD65; b) myelin basic protein; c) alpha synuclein; d) beta tau; e) type II collagen; f) myelin oligodendrocyte protein; and g) myosin.

9. The method of claim 1, wherein said tolerogenic agent and said antigen are administered using an oscillating needle and administering said tolerogenic agent and concurrently or subsequently said antigen with the oscillating needle into a layer of skin, wherein the layer of skin is dermal, epidermal, or subcutaneous tissue.

10. The method of claim 1, wherein the time interval between injection of said tolerogenic agent and said antigen is between 1 hour to 14 days.

11. The method of claim 1 wherein the surface area of administration is more than 1 cm(2).

12. The method of claim 1 wherein the area of administration is located near or on one or more lymph nodes.
